(19) United States
(12) Patent Application Publication
(10) Pub. No.: US 2007/0021762 A1
(43) Pub. Date: Jan. 25, 2007

(54) Ocular plug formed from placenta derived collagen biofabric

(76) Inventors: Qing Liu, Hillsborough, NJ (US); Cynthia Denice Ray, Branchburg, NJ (US)

Correspondence Address: JONES DAY, 222 EAST 41ST ST, NEW YORK, NY, 10017, US

(21) Appl. No.: 11/485,842

(22) Filed: Jul. 12, 2006

Related U.S. Application Data
(60) Provisional application No. 60/699,440, filed on Jul. 13, 2005.

(51) Int. Cl. A61F 9/00; A61F 2/14

(52) U.S. Cl. 606/157; 623/4.1; 435/379

(57) ABSTRACT

The present invention relates to ocular plugs formed from a biodegradable material. The plugs comprises a shaft and, optionally, a cap. The ocular plugs are intended to occlude, and to repair, discontinuities in the sclera, whether formed deliberately during injection or surgical foray into the eye, or accidentally. The method further provides methods of making the ocular plug. the invention also provides methods of using the ocular plugs to occlude and repair discontinuities in the sclera, or to deliver biologically active compounds to the sclera or the eye. Finally, the invention provides kits comprising one or more ocular plugs in a container.

This application claims benefit of U.S. Provisional Application Ser. No. 60/699,440, filed Jul. 13, 2006, which is hereby incorporated by reference herein in its entirety.

### 1. FIELD OF THE INVENTION

The present invention relates to an ocular plug comprising a medically useful biodegradable material, preferably a collagen biofabric produced from amnion and/or chorion. In one embodiment, the collagen biofabric, before forming into an ocular plug, has the structural integrity of the native non-treated amniotic membrane, i.e., substantially the native tertiary and quaternary collagen structure of amniotic membrane. The present invention also provides a method for preparing an ocular plug made from the collagen biofabric from a placental membrane, preferably a chorionic and/or amniotic membrane. The invention further provides methods of forming and making the ocular plug, and methods of using the ocular plug to occlude scleral holes and/or deliver bioactive compounds to the eye.

### 2. BACKGROUND OF THE INVENTION

2.1 Ocular Surgery

Vitreo-retinal surgery allows ophthalmologists to treat or repair disease or injuries to the posterior portion of the eye. In a typical surgery, various instruments are introduced into the vitreous cavity of the eye through one or more small access holes, typically in the area of the pars plana ciliaris. Such access holes, when not in use, or at the completion of surgery, must be plugged in some way to maintain the appropriate intraocular pressure, and to prevent egress of ocular fluid and the entrance of bacteria or other pathogens, or debris.

Moreover, it is frequently necessary to inject a compound directly to the interior of the eye by injecting the compound, typically using a syringe forced through the sclera. Such injections also leave holes in the sclera that must be closed or occluded to prevent leakage and infection.

With respect to surgery, typically, when surgery is complete, access holes are closed with sutures; however, such sutures are uncomfortable, and their insertion into the cornea risks infection and inappropriate proliferation of corneal cells in response to the damage caused by the suturing.

In the past, small steel plugs, having a straight shaft and a cap at one end, have been used to occlude the sites when instruments are not inserted through the holes. Ocular plugs having bulbous, rather than straight, shafts, are described in U.S. Pat. No. 6,846,318. Further, repeated insertion of surgical or other instruments into access holes generally results in the deformation and widening of the access hole. When this occurs, some ocular plug designs are unable to fit tightly into the access hole, allowing fluid leakage and invasion by outside agents. Moreover, known plugs do not contribute in any significant way to healing of the access holes.

Therefore, there is a need in the art for improved ocular plugs.

### 3. SUMMARY OF THE INVENTION

It is an object of the present invention to provide an ocular plug comprising a biodegradable material. The ocular plugs of the invention can, for example, be used for the occlusion of ocular holes formed at injection or surgical sites. Preferably, the ocular plug is formed from a biodegradable material, preferably collagen, particularly a collagen biofabric derived from a post-partum mammalian placenta, for example, from an amniotic membrane of the placenta.

In one embodiment, the present invention provides a plug comprising a cap and a shaft, the shaft having a surface, a length and two ends, said shaft extending from the cap, wherein said plug is made of a biodegradable composition. In a specific embodiment, said biodegradable composition comprises collagen. In a more specific embodiment, said collagen is derived from post-partum mammalian placenta. In another more specific embodiment, said composition is derived from amniotic membrane. In a specific embodiment, said shaft comprises a narrow portion and a wide portion, wherein said wide portion has a greater cross-sectional area than said narrow portion. In a more specific embodiment, said narrow portion is proximal to said cap, and said wide portion is distal to said cap. In another more specific embodiment, said wide portion is disposed on said shaft between said ends. In another more specific embodiment, said shaft has substantially equal cross-sectional area along its length. In a more specific embodiment, the surface of said shaft is knurled. In an even more specific embodiment, said shaft is knurled in a manner that facilitates placement of the plug into an ocular hole, and discourages removal of said plug from said ocular hole. In another more specific embodiment, the surface of said shaft is ribbed. In another more specific embodiment, said cross-sectional area increases substantially continuously from said narrow portion to said wide portion. In another more specific embodiment, said cross-sectional area increases substantially discontinuously from said narrow portion to said wide portion. In another specific embodiment, said plug creates a substantially watertight seal when placed into an ocular hole. In another specific embodiment, said shaft is of a sufficient diameter to seal a hole created by a 30 gauge or larger needle.

In another specific example, said collagen is obtained from an amniotic membrane. In another specific example, said shaft is solid. In another specific example, said cap is substantially flat. In another specific example, said plug is adapted for insertion into a hole in the sclera made as part of vitreo-retinal surgery or made by a needle. In another specific example, said plug comprises one or more growth factors or cytokines. In another specific example, said plug comprises a compound that inhibits the growth of, or kills, one or more microorganisms. In another specific example, said plug is coated with a tissue adhesive. In another specific example of the ocular plug of the invention, said collagen is obtained from a placenta. In another specific example, the collagen is obtained from an amniotic membrane.

In some embodiments, the ocular plug further comprises one or more biomolecules, e.g., therapeutic agents, including but not limited to, antibiotics, hormones, growth factors, anti-tumor agents, anti-fungal agents, anti-viral agents, pain medications, anti-histamines, anti-inflammatory agents, anti-infectives, wound healing agents, wound sealants, cellular attractants and scaffolding reagents, and the like. In a specific example, the collagen biofabric may be coated with or impregnated with one or more growth factors, for example, fibroblast growth factor, epithelial growth factor, etc. The collagen biofabric may be coated with or impregnated with one or more small molecules, including but not limited to small organic molecules such as specific inhibitors of particular biochemical processes e.g., membrane receptor inhibitors, kinase inhibitors, growth inhibitors, anti-cancer drugs, antibiotics, etc. In some embodiments, the collagen biofabric is coated with or impregnated with a biomolecule, during production or during preparation for surgery depending on its intended use.

The present invention further provides a method of making an ocular plug, comprising: (a) micronizing a dried amniotic membrane to produce micronized amniotic membrane; (b) forming said micronized amniotic membrane in a mold to produce an amniotic membrane plug; (c) freeze-drying said amniotic membrane plug to substantial dryness; and (d) crosslinking said amniotic membrane plug to form an ocular plug. In a specific embodiment, said micronizing is performed using a blender. In a more specific embodiment, the median size of particles in said micronized amniotic membrane is 1 micron to 1 mm. In another specific embodiment of the method, said freeze drying reduces the water content of said amniotic membrane plug to 20% or less by weight. In another specific embodiment, said crosslinking is performed using radiation. In a more specific embodiment of the method, said radiation is e-beam radiation, gamma radiation or ultraviolet radiation. In another more specific embodiment of the invention, said crosslinking is performed chemically. In another more specific embodiment of the invention, said crosslinking is performed using heat. In a more specific embodiment, said freeze dried amniotic membrane plug is treated in a vacuum oven at 105° C. for a time sufficient to achieve crosslinking.

The invention further comprises kits providing one or more of the ocular plugs of the invention in a suitable container. Kits of the invention comprise one or more ocular plugs, and may comprise other components, such as an instrument for inserting the ocular plug into the sclera, one or more bioactive compounds in one or more separate containers, one or more syringes, sterile gauze, gloves or other disposables, and the like.

The present invention further provides methods of repairing a discontinuity in the sclera of the eye comprising occluding the discontinuity with one or more ocular plugs of the invention. In one embodiment, the discontinuity is intentionally made. In a specific embodiment, the discontinuity is an injection site. In another specific embodiment, the discontinuity is a hole created in the sclera to allow the passage of a surgical instrument. In another embodiment, the discontinuity is caused by an accident, blow, or trauma.

3.1 Definitions

As used herein, “collagen biofabric” generally means a collagen-containing, placenta-derived amniotic and/or chorion membrane material used as a film, membrane, or sheet. A preferred collagen biofabric is the vacuum-dried, non-fixed, non-protease-treated amniotic membrane material described in Hariri, U.S. Application Publication U.S. 2004/0048796, which is hereby incorporated in its entirety, and produced by the methods described therein, herein (see Examples 1, 2). The collagen biofabric is preferably made from the amnion, but may be made from the chorion, or both amnion and chorion.

As used herein, the term “bioactive compound” means any compound or molecule that causes a measurable effect on one or more biological systems in vitro or in vivo.

### 4. BRIEF DESCRIPTION OF THE FIGURES

FIG. 1 One embodiment of an ocular plug, 10, comprising a shaft 20 that comprises a distal end 28, and, optionally, a cap 30 that comprises an upper face, 35 and a lower face, 37.

FIG. 2 One embodiment of the ocular plug showing a cap 30 comprising flanges 40.

FIG. 3 Ocular plugs in which the shaft 20 comprises a narrow portion 50 and a wide portion 60; (A) Embodiment of the ocular plug wherein the shaft flares continuously along the entire length. (B) Embodiment of the ocular plug wherein the shaft flares continuously along part of the length of the shaft.

FIGS. 4A-4C Embodiments of the ocular plug showing variations of the wide portion of the shaft used to anchor the plug in the sclera.

### 5. DETAILED DESCRIPTION OF THE INVENTION

The present invention provides an ocular plug made from a biodegradable material, preferably a collagen biofabric derived from the amnion, chorion, or both, of a mammalian placenta, preferably a human placenta. In addition to the ocular plug, the present invention also provides methods of making the ocular, and of using the ocular in a medical setting, e.g., occlusion of discontinuities, such as holes, in the sclera, and delivery of drugs to the sclera or to any part of the eye, e.g., interior or the eye.

5.1 Configurations

The ocular plugs of the present invention may be configured in any shape to accomplish the particular purpose at hand, e.g., occluding injection or ocular surgery-related holes in the sclera, prevention of leakage, drug delivery, anchoring of the plug, etc.

5.1.1 Plugs with Caps

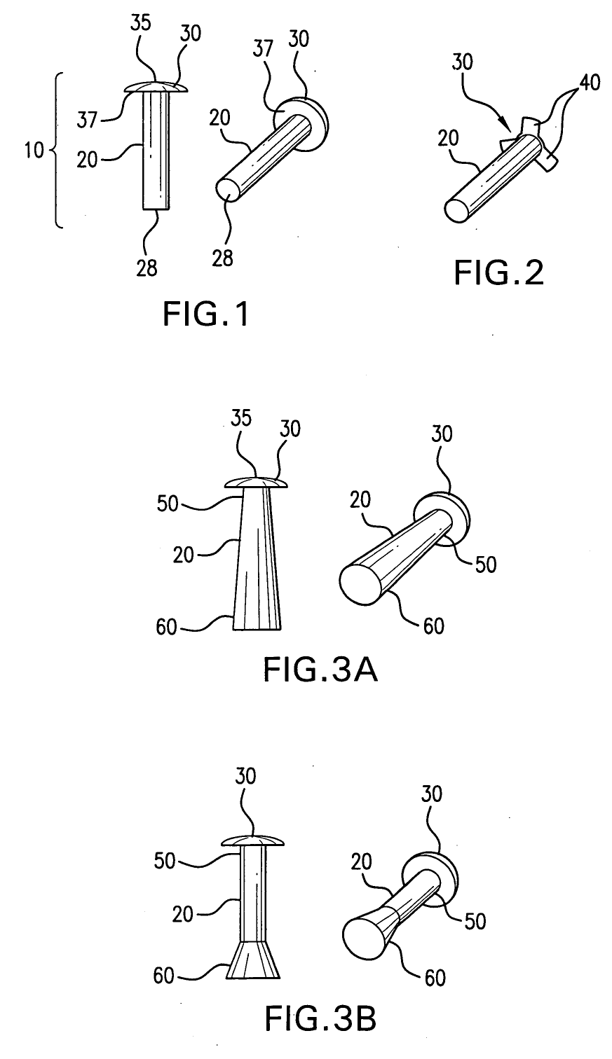

In one, preferred, embodiment, the invention provides an ocular plug 10 that comprises a shaft 20 attached to and extending from a cap 30, as shown in FIG. 1. Typically, the cap is circular when viewed from the upper face, 35. However, the cap may be oval, square, rectangular, polygonal, irregular, or may appear as a plurality of flanges 40 extending substantially perpendicularly from the shaft, as shown in FIG. 2. The upper face of the cap, 35, distal to the shaft, may be hemispherical, curved to a degree other than completely hemispherical, or may be substantially flat. Preferably, the surface of the upper face of the cap is shaped to approximate the curvature of the eye to promote comfort and reduce the possibility of inflammation or irritation associated with the eyelid moving over the face of the cap. The lower face of the cap, 37, proximal to the shaft, may be substantially flat, but is preferably shaped to approximate the curvature of the eye. Preferably, the cap tapers towards the edges so that a smooth transition is made from sclera to cap when the eyelid passes over the cap. However, the cap need not taper from center towards the edges, and may have a discernibly blunt edge.

The cap is preferably of a sufficient diameter to promote seating and maintenance of position of the plug within the hole in the sclera, and to reduce the possibility of the shaft from passing completely through the sclera during or after insertion of the plug into the sclera. The outer diameter of the cap may be from 1-10 times the diameter of the shaft; preferably, the outer diameter of the cap is between 1-3 times the diameter of the shaft.

The shaft, as the remainder of the plug, may be configured to accomplish occlusion of an injection- or ocular surgery-related scleral hole. The shaft may be thin enough, for example, to occlude the hole made by a 33 gauge, or thinner, needle after intravitreous injection, or may be as thick as 1-2 mm in diameter, or more, to occlude holes created during, for example, macular hole surgery. The shaft may be of any size appropriate to occlude a particular discontinuity in the sclera. The shaft is preferably at least as long as a sclera is thick, but may be shorter than the thickness of a sclera, or may be longer. A typical sclera is 0.35-0.55 mm thick, but may be thicker or thinner. The thickness depends upon the particular individual, as well as the position of the discontinuity in the sclera; for example, the sclera tends to thin away from the iris and towards the retina. Where the shaft is longer than the thickness of a sclera, the shaft, when the plug comprising it is fully inserted, projects through the sclera an into the vitreous humor.

The surface of the shaft may be smooth or textured. For example, the surface of the shaft may be rough, ribbed or knurled so as to enhance contact between the plug and sclera, thereby reducing the potential for the plug to work its way out of the scleral hole. Particularly where the plug comprises a cap, the shaft may be ribbed or knurled directionally; that is, ribbed or knurled to promote insertion of the plug into the scleral hole and to discourage passage of the plug in the opposite direction, i.e., back out of the scleral hole.

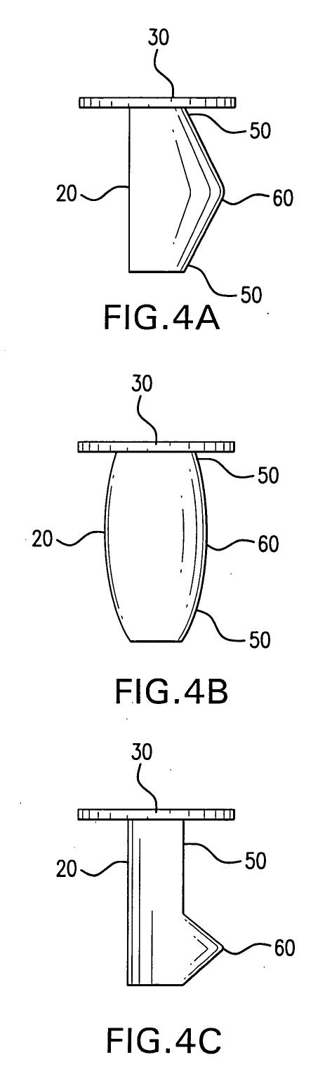

In a preferred embodiment, the shaft is substantially cylindrical. In another embodiment, the shaft is substantially cylindrical along its entire length. In other embodiments, the shaft is ovoid, square, rectangular, square or rectangular with rounded edges, polygonal, or irregular in cross-section. In another embodiment, the shaft comprises a narrow portion 50 and a wide portion 60. See FIGS. 3A-3B, 4A-4C. Typically, the shaft is attached to the cap through the narrow portion; the wide portion, distal to the cap, facilitates anchoring of the plug into the sclera. In one embodiment, the cross-sectional area of the wide portion is greater than that of the narrow portion. The wide portion of the shaft may be manufactured in a variety of configurations. For example, the shaft may flare, as shown in FIGS. 3A and 3B. Such a flare may be substantially continuous along the length of the shaft, or may begin at any point along the length of the shaft. In another embodiment, the wide portion is a flange or protrusion from a portion of the main body of the shaft, e.g., from one side of the shaft, as shown in FIGS. 4A-4C. Such a flange or protrusion may have any shape that facilitates maintenance of the plug within the scleral hole while not substantially increasing the difficulty of insertion or the potential for scleral damage during insertion. In another embodiment, the wide portion comprises a flange or other protrusion that substantially encircles the shaft. For example, as shown in FIGS. 3A-3B, the wide portion may be an inverted cone or frustum, wherein the larger radius of the frustum is wider than the diameter of the shaft. In another embodiment, the wider portion of the shaft is a cylinder having a radius larger than the radius of the shaft. In another embodiment, the wider portion of the shaft has substantially the same cross-sectional shape as the shaft, but a cross-sectional area larger than the cross-sectional area of the shaft. There is no need, however, for the wide portion of the shaft to have a particular shape relative to the cross-sectional shape of the shaft, and the wide portion need not have the same cross-sectional shape as the shaft. In another embodiment, the shaft comprises a thread spirally disposed along a portion or all of the length of the shaft, so that the shaft of the plug functions as a screw. In this embodiment, the thread may proceed clockwise or counterclockwise along the shaft.

In another embodiment, the plug may be constructed so that the portion of the shaft distal to the cap comprises one or more flaps that may be folded against the shaft during insertion of the plug into the sclera, and which open, or fold away, from the shaft once the flap has been pushed completely through the sclera. The one or more flaps would act as an anchor.

In one embodiment, the wider portion of the shaft extends into the sclera itself, and serves as an anchor. In another embodiment, part or all of the wide portion of the shaft extends into the vitreous humor.

The end of the shaft distal to the cap, 28 (FIG. 1), may be flat, rounded, or tapered, or may be irregular. The surface of the end may be substantially perpendicular to the longitudinal axis of the shaft, or may be tilted, giving the end of the shaft a barbed appearance.

In one embodiment, the ocular plug comprises an opening extending at least the portion of the cap distal to the shaft, and, optionally, into the shaft. The opening can be used, for example, to receive a wire of fixed gauge. The wire is used to pick up the ocular plug and guide the ocular plug into a scleral discontinuity.

5.1.2 Plugs Without Caps

The ocular plug of the invention need not comprise a cap. For example, the ocular plug may comprise a shaft only. In this embodiment, the shaft may be formed in any of the configurations as for the shafts of a plug with a cap, as in Section 5.1.1, above. For example, in its simplest form, the plug may simply be a cylinder, with a smooth, ribbed, knurled or textured surface, or may comprise one or more wide portions that can act as anchors. In one embodiment, the shaft (that is, the plug) comprises two wide portions. In a specific embodiment, the shaft is dumbbell-shaped. The dumbbell shape may be accomplished, for example, by thickening the ends of the shaft so that the change in thickness from center of the shaft to either end is continuous; alternatively, the change in thickness from center of the shaft to either end is discontinuous. Preferably, in this embodiment, the length of the shaft between the wide portions (e.g., the ends of the dumbbell) is at least the thickness of the sclera.

5.1.3 Dimensions

The ocular plugs of the invention may be pre-made to standard sizes, or may be custom-made to fill particular scleral holes or discontinuities, whether anticipated (as in the case of surgery) or unanticipated. In one embodiment, therefore, the invention provides an ocular plug, wherein said ocular plug has a shaft of a reproducible, standard size. The ocular plug may also be custom-made for a particular discontinuity. In specific embodiments, the standard or custom-made diameter size of a shaft for said ocular plug is a diameter sufficient to substantially occlude a scleral hole caused by passage of a 33, 32, 31, 30, 29, 28, 27, 26, 25, 24, 23, 22, 21, 20, 19, 18, 17, 16, 15, 14, 13, 12 or larger gauge needle. In other specific embodiments, the standard or custom-made diameter of a shaft for said ocular plug is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 millimeters, or wider. In other specific embodiments, the standard length of the shaft of said plug is 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45 or 1.50 millimeters.

5.2 Materials

The ocular plug of the present invention is made primarily, or exclusively, of a biodegradable composition. The biodegradable composition is preferably suitable for use in a medical-setting to, e.g., place in the sclera, or other bodily tissue. In particular, “biodegradable” in this context indicates that the composition may be broken down or assimilated by the patient or individual receiving the biodegradable material. Such biodegradable compositions include, for example, biodegradable polymers from natural sources and synthetic biodegradable polymers. Synthetic biodegradable compositions suitable for forming or making the ocular plug of the present invention include, but are not limited to, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(DTH iminocarbonate), poly(bisphenol A iminocarbonate), poly(ortho ester), polycyanoacrylate, and polyphosphazene. Biodegradable compositions from natural sources include, but are not limited to, modified polysaccharides, such as cellulose, chitin, or dextran; modified proteins, such as fibrin or casein; or collagen-based materials, such as those derived from amniotic membrane from post-partum mammalian placentas. A preferred material for the production of the ocular plugs disclosed herein is dried amniotic membrane from postpartum mammalian placenta, and particularly is the collagen biofabric described in U.S. Application Publication No. 2004/0048796, which is hereby incorporated in its entirety.

5.2.1 Collagen Biofabric

The ocular plug of the present invention is preferably formed or made from a collagen biofabric. The collagen biofabric may be derived from the amniotic membrane of any mammal, for example, equine, bovine, porcine or catarrhine sources, but is most preferably derived from human placenta. In a preferred embodiment, the collagen biofabric is substantially dry, i.e., is 20% or less water by weight. In another preferred embodiment, the collagen biofabric retains the native tertiary and quaternary structure of the amniotic membrane, i.e., has not been protease-treated. In another preferred embodiment, the collagen biofabric, prior to forming the ocular plug as described below, contains no collagen and other structural proteins that have been artificially crosslinked, e.g., chemically crosslinked, that is, the preferred collagen biofabric is not fixed prior to formation of the ocular plug. A preferred collagen biofabric is the dried, non-fixed, non-protease-treated amniotic membrane material described in Hariri, U.S. Application Publication U.S. 2004/0048796, which is hereby incorporated in its entirety, and that is produced by the methods described therein, and herein (see Examples 1, 2). However, the methods of the present invention can utilize any placenta-derived collagen material made by any procedure.

In one embodiment, the collagen biofabric used in the ocular plug of the invention is translucent. In other embodiments, the collagen biofabric is opaque, or is colored or dyed, e.g., permanently colored or dyed, using a medically-acceptable dyeing or coloring agent; such an agent may be adsorbed onto the collagen biofabric, or the collagen biofabric may be impregnated or coated with such an agent. In this embodiment, any known non-toxic, non-irritating coloring agent or dye may be used.

When the collagen biofabric is substantially dry, it is about 0.1 g/cm² to about 0.6 g/cm². In a specific embodiment, a single layer of the collagen biofabric is at least 2 microns in thickness. In another specific embodiment, a single layer of the collagen biofabric used to form an ocular plug is approximately 10-40 microns in thickness, but may be approximately 2-150, 2-100 microns, 5-75 microns or 7-60 microns in thickness in the dry state.

In one embodiment, the collagen biofabric is principally comprised of collagen (types I, III and IV; about 90% of the matrix of the biofabric), fibrin, fibronectin, elastin, and may further comprise glycosaminoglycans and/or proteoglycans. In certain embodiments, the collagen biofabric can comprise non-structural components, such as, for example, one or more growth factors, e.g., platelet-derived growth factors (PDGFs), vascular-endothelial growth factor (VEGF), fibroblast growth factor (FGF) and transforming growth factor-β1. The composition of the collagen biofabric may thus be ideally suited to encourage the migration of fibroblasts and macrophages, and thus the promotion of wound healing.

The collagen biofabric used to make the ocular plug may be single-layered, for example, a single-layer sheet or an un-laminated membrane.

The collagen biofabric used to form the ocular plug of the invention may further comprise collagen from a non-placenta source. For example, one or more layers of collagen biofabric may be coated or impregnated with, or layered with, purified extracted collagen. Such collagen may be obtained, for example, from commercial sources, or may be produced according to known methods, such as those disclosed in U.S. Pat. Nos. 4,420,339; 5,814,328; and 5,436,135, the disclosures of which are hereby incorporated by reference.

The collagen biofabric used to form the ocular plug of the present invention may comprise one or more compounds or substances that are not present in the placental material from which the collagen biofabric is derived. For example, the collagen biofabric may be coated with or impregnated with, before or after forming the ocular plug, a bioactive compound. Such bioactive compounds include, but are not limited to, small organic molecules (e.g., drugs), antibiotics (such as Tetracycline, Clindamycin, Minocycline, Doxycycline, Gentamycin), hormones, growth factors, anti-tumor agents, anti-fungal agents, anti-viral agents, pain medications, anti-histamines, anti-inflammatory agents, anti-infectives including but not limited to silver (such as silver salts, including but not limited to silver nitrate and silver sulfadiazine), elemental silver, antibiotics, bactericidal enzymes (such as lysozyme), wound healing agents (such as cytokines including but not limited to PDGF, TGF; thymosin), hyaluronic acid as a wound healing agent, wound sealants (such as fibrin with or without thrombin), cellular attractant and scaffolding reagents (such as added fibronectin) and the like. In a specific example, the collagen biofabric may be impregnated with at least one growth factor, for example, fibroblast growth factor, epithelial growth factor, etc. The biofabric may also be impregnated with small organic molecules such as specific inhibitors of particular biochemical processes e.g., membrane receptor inhibitors, kinase inhibitors, growth inhibitors, anticancer drugs, antibiotics, etc. Impregnating the collagen biofabric with a bioactive compound may be accomplished, e.g., by immersing the collagen biofabric in a solution of the bioactive compound of the desired concentration for a time sufficient to allow the collagen biofabric to absorb and to equilibrate with the solution; by spraying the solution onto the biofabric; by wetting the biofabric with the solution, etc.

In other embodiments, the collagen biofabric may be combined with a hydrogel. Preferably, the collagen biofabric is combined with a hydrogel after the ocular plug is formed. Any hydrogel composition known to one skilled in the art is encompassed within the invention, e.g., any of the hydrogel compositions disclosed in the following reviews: Graham, 1998, Med. Device Technol. 9(1): 18-22; Peppas et al., 2000, Eur. J. Pharm. Biopharm. 50(1): 27-46; Nguyen et al., 2002, Biomaterials, 23(22): 4307-14; Henincl et al., 2002, Adv. Drug Deliv. Rev 54(1): 13-36; Skelhorne et al., 2002, Med. Device. Technol. 13(9): 19-23; Schmedlen et al., 2002, Biomaterials 23: 4325-32; all of which are incorporated herein by reference in their entirety. In a specific embodiment, the hydrogel composition is applied on the collagen biofabric, i.e., disposed on the surface of the collagen biofabric. The hydrogel composition for example, may be sprayed onto the collagen biofabric or coated onto the surface of the collagen biofabric, or the biofabric may be soaked, bathed or saturated with the hydrogel composition. In another specific embodiment, the hydrogel is sandwiched between two or more layers of collagen biofabric. In an even more specific embodiment, the hydrogel is sandwiched between two or more layers of collagen biofabric, wherein the edges of the two layers of biofabric are sealed so as to substantially or completely contain the hydrogel.

The hydrogels useful in the methods and compositions of the invention can be made from any water-interactive, or water soluble polymer known in the art, including but not limited to, polyvinylalcohol (PVA), polyhydroxyehthyl methacrylate, polyethylene glycol, polyvinyl pyrrolidone, hyaluronic acid, alginate, collagen, gelatin, dextran or derivatives and analogs thereof.

In some embodiments, the collagen biofabric of the invention comprises one or more bioactive compounds and is combined with a hydrogel. For example, the collagen biofabric can be impregnated with one or more bioactive compounds prior to being combined with a hydrogel. In other embodiments, the hydrogel composition is further impregnated with one or more bioactive compounds prior to, or after, being combined with a collagen biofabric of the invention, for example, the bioactive compounds described in Section 5.2.1.1, below.

5.2.1.1 Bioactive Compounds

The collagen biofabric used in the methods of the invention may comprise (e.g., be impregnated with or coated with) one or more bioactive or medicinal compounds, such as small organic molecules (e.g., drugs), antibiotics, antiviral agents, antimicrobial agents, anti-inflammatory agents, antiproliferative agents, cytokines, enzyme or protein inhibitors, antihistamines, and the like. In various embodiments, the collagen biofabric may be coated or impregnated with antibiotics (such as Clindamycin, Minocycline, Doxycycline, Gentamycin), hormones, growth factors, anti-tumor agents, anti-fungal agents, anti-viral agents, pain medications (including Xylocaine®, Lidocaine, Procaine, Novocaine, etc.), antihistamines (e.g., diphenhydramine, Benadryl®, etc.), anti-inflammatory agents (e.g. steroids, NSAIDs, Xibrom, diclofenac, nefanac, etc.), anti-infectives including but not limited to silver (such as silver salts, including but not limited to silver nitrate and silver sulfadiazine), elemental silver, antibiotics, bactericidal enzymes (such as lysozome), wound healing agents (such as cytokines including but not limited to PDGF (e.g., REGRANEX®), TGF; thymosin), hyaluronic acid as a wound healing agent, wound sealants (such as fibrin with or without thrombin), cellular attractant and scaffolding reagents (such as fibronectin), and the like, or combinations of any of the foregoing, or of the foregoing and other compounds not listed. Such impregnation or coating may be accomplished by any method known in the art, and a portion or the whole of the collagen biofabric may be so coated or impregnated.

The collagen biofabric, or composition comprising collagen biofabric, may comprise any of the compounds listed herein, without limitation, individually or in any combination. Any of the biologically active compounds listed herein, and others useful in the context of the sclera or eye, may be formulated by known methods for immediate release or extended release. Additionally, the collagen biofabric may comprise two or more biologically active compounds in different manners; e.g., the biofabric may be impregnated with one biologically active compound and coated with another. In another embodiment, the collagen biofabric comprises one biologically active compound formulated for extended release, and a second biologically active compound formulated for immediate release.

Wound healing, including the healing of scleral discontinuities, requires adequate nutrition, particularly the presence of iron, zinc, arginine, vitamin C, arginine, and the like. Thus, the collagen biofabric may be impregnated or coated with a physiologically-available form of one or more nutrients required for wound healing. Preferably, the nutrient is formulated for extended release.

The collagen biofabric, or composition comprising collagen biofabric, may comprise an antibiotic. In certain embodiments, the antibiotic is a macrolide (e.g., tobramycin (Tobi®)), a cephalosporin (e.g., cephalexin (Keflex®)), cephradine (Velosef®)), cefuroxime (Ceftin®, cefprozil (Cefzil®), cefaclor (Ceclor®), cefixime (Suprax® or cefadroxil (Duricef®), a clarithromycin (e.g., clarithromycin (Biaxin)), an erythromycin (e.g., erythromycin (EMycin®)), a penicillin (e.g., penicillin V (V-CillinK® or Pen VeeK®)) or a quinolone (e.g., ofloxacin (Floxin®), ciprofloxacin (Cipro®) ornorfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In certain embodiments, the collagen biofabric may be coated or impregnated with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

In certain other embodiments, the collagen biofabric, or a composition comprising collagen biofabric, is coated or impregnated with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, methotrexate, colchicine, allopurinol, probenecid, sulfinpyrazone and benzbromarone.

In certain embodiments, the collagen biofabric, or a composition comprising collagen biofabric, is coated or impregnated with an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

The collagen biofabric, or a composition comprising collagen biofabric, may also be coated or impregnated with a cytokine receptor modulator. Examples of cytokine receptor modulators include, but are not limited to, soluble cytokine receptors (e.g., the extracellular domain of a TNF-α receptor or a fragment thereof, the extracellular domain of an IL-10 receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-10 antibodies, anti-IL-6 antibodies, anti-IL-8 antibodies (e.g., ABX-IL-8 (Abgenix)), and anti-IL-12 antibodies). In a specific embodiment, a cytokine receptor modulator is IL-4, IL-10, or a fragment thereof. In another embodiment, a cytokine receptor modulator is an anti-IL-1 antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

The collagen biofabric, or a composition comprising collagen biofabric, may also be coated or impregnated with a cytokine. Examples of cytokines include, but are not limited to, colony stimulating factor 1 (CSF-1), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), insulin-like growth factor 1 (IGF-1), platelet derived growth factor (PDGF), erythropoietin (Epo), epidermal growth factor (EGF), fibroblast growth factor (FGF) (basic or acidic), granulocyte macrophage stimulating factor (GM-CSF), granulocyte colony stimulating factor (G-CSF), heparin binding epidermal growth factor (HEGF), macrophage colony stimulating factor (M-CSF), prolactin, and interferon (IFN), e.g., IFN-alpha, and IFN-gamma), transforming growth factor alpha (TGF-α), TGFβ1, TGFβ2, tumor necrosis factor alpha (TNF-α), vascular endothelial growth factor (VEGF), hepatocyte growth factor (HGF), etc.

The collagen biofabric may also be coated or impregnated with a hormone. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins. Examples of β-interferons include, but are not limited to, interferon β1-a and interferon β1-b.

The collagen biofabric, or composition comprising collagen biofabric, may also be coated or impregnated with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The collagen biofabric, or a composition comprising collagen biofabric, may also be coated or impregnated with an immunomodulatory agent, including but not limited to methothrexate, leflunomide, cyclophosphamide, cyclosporine A, macrolide antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 antibodies (e.g., cM-T412 (Boeringer), IDEC-CE9.Is (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD1 1a antibodies (e.g., Xanelim (Genentech)), and anti-B7 antibodies (e.g., IDEC-114) (IDEC))) and CTLA4-immunoglobulin. In a specific embodiment, a T cell receptor modulator is a CD2 antagonist. In other embodiments, a T cell receptor modulator is not a CD2 antagonist. In another specific embodiment, a T cell receptor modulator is a CD2 binding molecule, preferably MEDI-507. In other embodiments, a T cell receptor modulator is not a CD2 binding molecule.

The collagen biofabric, or composition comprising collagen biofabric, may also be coated or impregnated with a class of immunomodulatory compounds known as IMiDs®. As used herein and unless otherwise indicated, the term “IMiD®” and “IMiDs®” (Celgene Corporation) encompasses small organic molecules that markedly inhibit TNF-α, LPS induced monocyte IL1β and IL12, and partially inhibit IL6 production. Specific immunomodulatory compounds are discussed below.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl) phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. patent publication no. 2003/0096841 published on May 22, 2003, and International Application No. PCT/US01/50401 (International Publication No. WO 02/059106). The entireties of each of the patents and patent applications identified herein are incorporated herein by reference. Immunomodulatory compounds do not include thalidomide.

Various immunomodulatory compounds contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds may be used in methods and compositions. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

Preferred immunomodulatory compounds include, but are not limited to, 1-oxo-and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I: 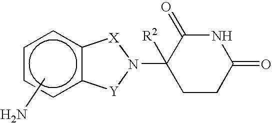
in which one of X and Y is C═O, the other of X and Y is C═O or CH₂, and R² is hydrogen or lower alkyl, in particular methyl. Specific immunomodulatory compounds include, but are not limited to: 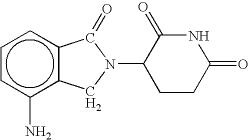
1-oxo-2-(2,6-dioxopiperidin-3-yl)-4-aminoisoindoline; 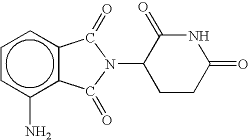
1,3-dioxo-2-(2,6-dioxopiperidin-3-yl)-4-aminoisoindoline; and 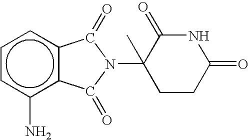
1,3-dioxo-2-(3-methyl-2,6-dioxopiperidin-3-yl)-4-aminoisoindole, and optically pure isomers thereof. The compounds can be obtained via standard, synthetic methods (see e.g., U.S. Pat. No. 5,635,517, incorporated herein by reference). The compounds are also available from Celgene Corporation, Warren, N.J.

As used herein, and unless otherwise indicated, the term “optically pure” means a composition that comprises one optical isomer of a compound and is substantially free of other isomers of that compound. For example, an optically pure composition of a compound having one chiral center will be substantially free of the opposite enantiomer of the compound. An optically pure composition of a compound having two chiral centers will be substantially free of other diastereomers of the compound. A typical optically pure compound comprises greater than about 80% by weight of one enantiomer of the compound and less than about 20% by weight of other enantiomers of the compound, more preferably greater than about 90% by weight of one enantiomer of the compound and less than about 10% by weight of the other enantiomers of the compound, even more preferably greater than about 95% by weight of one enantiomer of the compound and less than about 5% by weight of the other enantiomers of the compound, more preferably greater than about 97% by weight of one enantiomer of the compound and less than about 3% by weight of the other enantiomers of the compound, and most preferably greater than about 99% by weight of one enantiomer of the compound and less than about 1% by weight of the other enantiomers of the compound.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476;052, and International Patent Application No. PCT/US97/13375 (International Publication No. WO 98/03502), each of which is incorporated herein by reference. Representative compounds are of formula: 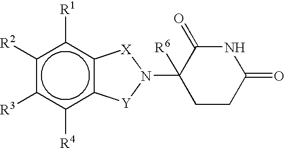
in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas: 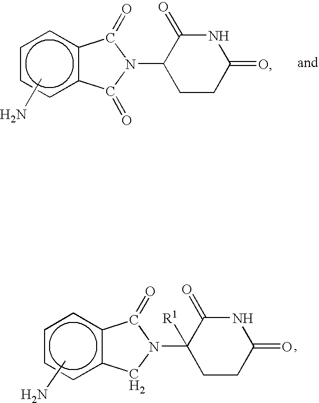
wherein R¹ is hydrogen or methyl. In a separate embodiment, the invention encompasses the use of enantiomerically pure forms (e.g. optically pure (R) or (S) enantiomers) of these compounds.

Still other specific immunomodulatory compounds belong to a class of isoindole-imides disclosed in U.S. Patent Application Publication Nos. US 2003/0096841 and US 2003/0045552, and International Application No. PCT/US01/50401 (International Publication No. WO 02/059106), each of which are incorporated herein by reference. Representative compounds are of formula II: 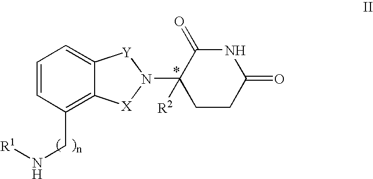
and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

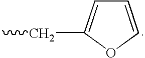

In another embodiment of the compounds of formula II, R¹ is

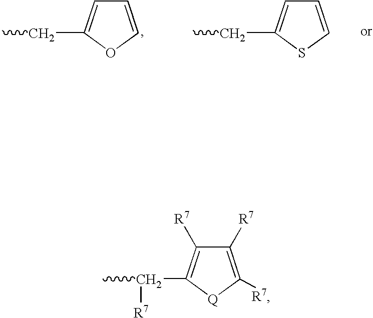

wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C0-C4)alkyl-C2-C5)heteroaryl, (C1-C8)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III: 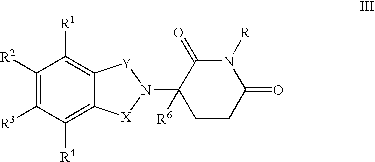
and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of 1 to 8 carbon atoms, or phenyl; and

* represents a chiral-carbon center.

Other representative compounds are of formula: 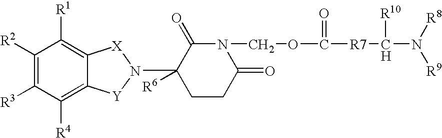
wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹H₂CH₂— in which X¹ is —O—, —S—, or —NH—; and

R¹⁰ is hydrogen, alkyl of to 8 carbon atoms, or phenyl.

Other representative compounds are of formula: 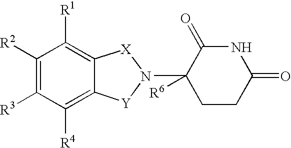
in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula: 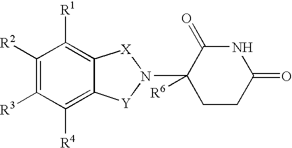
in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula: 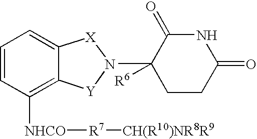
in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Preferred immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure:

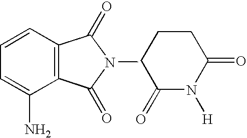

The compound 3-(4-amino-1-oxo-1,3-dihydro-isoindol-2-yl)-piperidine-2,6-dione has the following chemical structure:

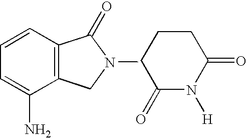

In another embodiment, specific immunomodulatory compounds encompass polymorphic forms of 3-(4-amino-1-oxo-1,3 dihydro-isoindol-2-yl)-piperidene-2,6-dione such as Form A, B, C, D, E, F, G and H, disclosed in U.S. provisional application No. 60/499,723 filed on Sep. 4, 2003, and U.S. non-provisional application Ser. No. 10/934,863, filed Sep. 3, 2004, both of which are incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C. Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula: 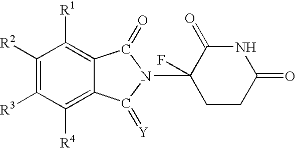
wherein:

Y is oxygen or H² and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds include, but are not limited to, the tetra substituted 2-(2,6-dioxopiperdin-3-yl)-1-oxoisoindolines described in U.S. Pat. No. 5,798,368, which is incorporated herein by reference. Representative compounds are of formula: 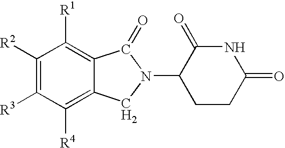
wherein each of R¹, R², R³, and R⁴, independently of the others, is halo, alkyl of 1 to 4 carbon atoms, or alkoxy of 1 to 4 carbon atoms.

Other specific immunomodulatory compounds include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula: 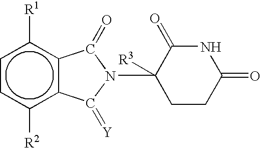
in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula: 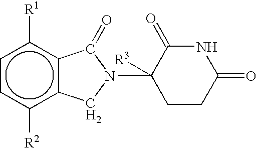
wherein

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula: 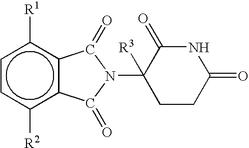
wherein:

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl.

Other specific immunomodulatory compounds include, but are not limited to, 1-oxo and 1,3-dioxoisoindolines substituted in the 4- or 5-position of the indoline ring described in U.S. Pat. No. 6,380,239 and co-pending U.S. application Ser. No. 10/900,270, filed Jul. 28, 2004, which are incorporated herein by reference. Representative compounds are of formula: 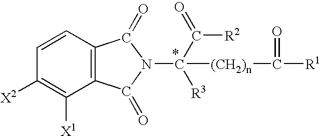
in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula: 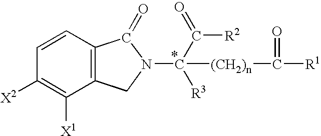
in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

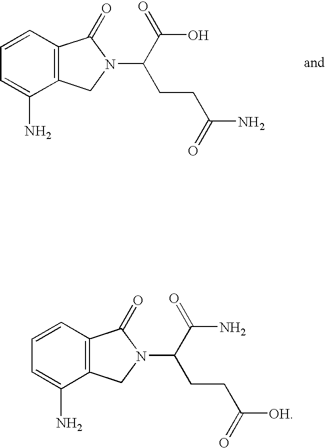

Other representative compounds are of formula: 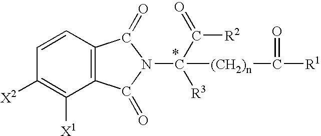
in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptablesalts, solvate, prodrugs, and stereoisomers thereof:

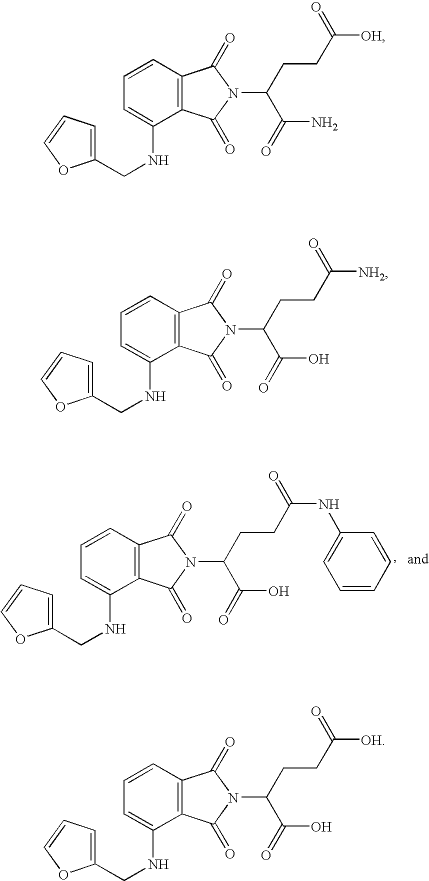

Other specific examples of the compounds are of formula: 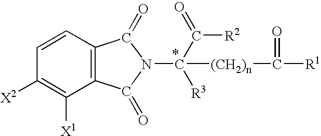
wherein:

one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula: 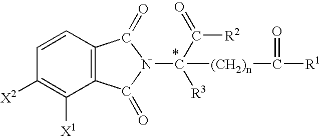
wherein:

one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula: 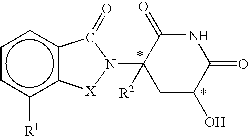
wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

The immunomodulatory compounds disclosed herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkloxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound of the invention is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds of this invention (e.g., R/S=30/70, 35/65, 40/60, 45/55, 55/45, 60/40, 65/35 and 70/30). Various immunomodulatory compounds of the invention contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

5.2.1.2 Method of Making Collagen Biofabric

Collagen biofabric, made from amniotic membrane, chorionic membrane, or both, may be produced by any means that preserves the biochemical and structural characteristics of the membrane's components—chiefly collagen, elastin, laminin, and fibronectin. A preferred material is the collagen biofabric described in, and produced according to the methods disclosed in, United States Application Publication No. U.S. 2004/0048796 A1, “Collagen Biofabric and Methods of Preparation and Use Therefor” by Hariri, which is hereby incorporated herein in its entirety.

Preferably, the collagen biofabric used to make an ocular plug is derived from a human placenta for use in human subjects, though the collagen biofabric may be made from amniotic membrane from a non-human mammal. Where the collagen biofabric is to be used in a non-human animal, it is preferred that the collagen biofabric be derived from a placenta from that species of animal

In a preferred embodiment, the placenta for use in the methods of the invention is taken as soon as possible after delivery of the newborn. The placenta may be used immediately, or may be stored for 2-5 days from the time of delivery prior to any further treatment. The placenta is typically exsanguinated, that is, drained of the cord blood remaining after birth. Preferably, the expectant mother is screened prior to the time of birth, using standard techniques known to one skilled in the art, for communicable diseases including but not limited to, HIV, HBV, HCV, HTLV, syphilis, CMV, and other viral pathogens known to contaminate placental tissue.

One exemplary method for preparing a collagen biofabric of the invention comprises the following steps:

Step I. The umbilical cord is separated from the placental disc; optionally, the amniotic membrane is separated from the chorionic membrane. In a preferred embodiment, the amniotic membrane is separated from the chorionic membrane prior to cutting the placental membrane. Following separation of the amniotic membrane from the chorionic membrane and placental disc, the umbilical cord stump is cut, e.g., with scissors, and detached from the placental disc. The amniotic membrane may then be stored in a sterile, preferably buffered, saline solution, such as 0.9% sterile NaCl solution. Preferably, the amniotic membrane is stored by refrigeration, at a temperature of at least 2° C.

Step II. The amniotic membrane is substantially decellularized; that is, substantially all cellular material and cellular debris (e.g., all visible cellular material and cellular debris) is removed. Any decellularizing process known to one skilled in the art may be used, however, generally the process used for decellularizing the amniotic membrane of the invention does not disrupt the native conformation of the proteins making up the biofabric. “Substantial decellularization” of the amniotic membrane preferably removes at least 90% of the cells, more preferably removes at least 95% of the cells, and most preferably removes at least 99% of the cells (e.g., fibroblasts, amniocytes and chorionocytes). The amniotic membranes decellularized in accordance with the methods of the invention are uniformly thin, with thickness variations of between about 2 and about 150 microns in the dry state, smooth (as determined by touch) and clear in appearance. Decellularization may comprise physical scraping, for example, with a sterile cell scraper, in combination with rinsing with a sterile solution. The decellularization technique employed should not result in gross disruption of the anatomy of the amniotic membrane or alter the biomechanical properties of the amniotic membrane. Preferably, the decellularization of the amniotic membrane comprises use of a detergent-containing solution, such as nonionic detergents, Triton X-100, anionic detergents, sodium dodecyl sulfate, Any mild anionic detergent, i.e., a non-caustic detergent, with a pH of 6 to 8, and low foaming, can be used to decellularize the amniotic membrane. In a specific embodiment, 0.01-1% deoxycholic acid sodium salt monohydrate is used in the decellularization of the amniotic membrane.

It is highly preferable to limit the protease activity in preparation of the biofabric. Additives to the lysis, rinse and storage solutions such as metal ion chelators, for example 1,10-phenanthroline and ethylenediaminetetraacetic acid (EDTA), create an environment unfavorable to many proteolytic enzymes. Providing sub-optimal conditions for proteases such as collagenase, assists in protecting amniotic membrane components such as collagen from degradation during the cell lysis step. Suboptimal conditions for proteases may be achieved by formulating the hypotonic lysis solution to eliminate or limit the amount of calcium and zinc ions available in solution. Many proteases are active in the presence of calcium and zinc ions and lose much of their activity in calcium and zinc ion free environments. Preferably, the hypotonic lysis solution will be prepared selecting conditions of pH, reduced availability of calcium and zinc ions, presence of metal ion chelators and the use of proteolytic inhibitors specific for collagenase such that the solution will optimally lyse the native cells while protecting the underlying amniotic membrane from adverse proteolytic degradation. For example a hypotonic lysis solution may include a buffered solution of water, pH 5.5 to 8, preferably pH 7 to 8, free from calcium and zinc ions and including a metal ion chelator such as EDTA. Additionally, control of the temperature and time parameters during the treatment of the amniotic membrane with the hypotonic lysis solution may also be employed to limit the activity of proteases.

It is preferred that the decellularization treatment of the amniotic membrane also limits the generation of new immunological sites. Since enzymatic degradation of collagen is believed to lead to heightened immunogenicity, the invention encompasses treatment of the amniotic membrane with enzymes, e.g., nucleases, that are effective in inhibiting cellular metabolism, protein production and cell division, that minimize proteolysis of the compositions of the amniotic membrane thus preserving the underlying architecture of the amniotic membrane. Examples of nucleases that can be used in accordance with the methods of the invention are those effective in digestion of native cell DNA and RNA including both exonucleases and endonucleases. A non-limiting example of nucleases that can be used in accordance with the methods of the invention include exonucleases that inhibit cellular activity, e.g., DNase I (SIGMA Chemical Company, St. Louis, Mo.) and RNase A (SIGMA Chemical Company, St. Louis, Mo.) and endonucleases that inhibit cellular activity, e.g., EcoRI (SIGMA Chemical Company, St. Louis, Mo.) and HindIll (SIGMA Chemical Company, St. Louis, Mo.). It is preferable that the selected nucleases are applied in a physiological buffer solution which contains ions, e.g., magnesium, calcium, which are optimal for the activity of the nuclease. Preferably, the ionic concentration of the buffered solution, the treatment temperature and the length of treatment are selected by one skilled in the art by routine experimentation to assure the desired level of nuclease activity. The buffer is preferably hypotonic to promote access of the nucleases to cell interiors.

In another embodiment of Steps I and II, above, the placenta, after initial processing, is briefly rinsed in saline to remove blood from the placental surface. The placental disk is then immersed in a cold deoxycholic acid solution at a concentration of about 0.1% to about 10%, and, in a specific embodiment, about 0.1% to about 2.0%. The placenta is then incubated in this solution at between about 1° C. to about 8° C. for about 5 days to about 6 months. In specific embodiments, the placental disk is immersed, for example, for about 5 to about 15 days; about 5 to about 30 days, about 5 to about 60 days, or for up to about one year. Typically, the deoxycholic acid solution is replaced during incubation every 2-5 days. In another specific embodiment, the placental disk is immersed in a deoxycholic acid solution at a concentration of about 1% at a temperature of 0° C. to about 8° C. for about 5 days to about 15 days. This incubation serves two purposes. First, it allows time for serological tests to be performed on the placental material and blood, so that placentas failing to meet serological criteria are not processed further. Second, the longer incubation improves the removal of epithelial cells and fibroblasts, which allows for a significant reduction in the amount of time spent decellularizing the amnion by physically scraping. Typically, the scraping time is reduced from, e.g., about 40 minutes to about 20 minutes. The amniotic membrane is then dried as described below.

Step III. Following decellularization, the amniotic membrane is washed to assure removal of cellular debris which may include cellular proteins, cellular lipids, and cellular nucleic acids, as well as any extracellular debris such as extracellular soluble proteins, lipids and proteoglycans. The wash solution may be de-ionized water or an aqueous hypotonic buffer. Preferably, the amniotic membrane is gently agitated for 15-120 minutes in the detergent, e.g., on a rocking platform, to assist in the decellularization. The amniotic membrane may, after detergent decellularization, again be physically decellularized as described above; the physical and detergent decellularization steps may be repeated as necessary, as long as the integrity of the amniotic membrane is maintained, until no visible cellular material and cellular debris remain.

In certain embodiments, the amniotic membrane is dried immediately (i.e., within 30 minutes) after the decellularization and washing steps. Alternatively, when further processing is not done immediately, the amniotic membrane may be refrigerated, e.g., stored at a temperature of about 1° C. to about 20° C., preferably from about 2° C. to about 8° C., for up to 28 days prior to drying. When the decellularized amniotic membrane is stored for more than three days but less than 28 days, the sterile solution covering the amniotic membrane is preferably changed periodically, e.g., every 1-3 days.

In certain embodiments, when the amniotic membrane is not refrigerated after washing, the amniotic membrane is washed at least 3 times prior to proceeding to Step IV of the preparation. In other embodiments, when the amniotic membrane has been refrigerated and the sterile solution has been changed once, the amniotic membrane is washed at least twice prior to proceeding to Step IV of the preparation. In yet other embodiments, when the amniotic membrane has been refrigerated and the sterile solution has been changed twice or more, the amniotic membrane is washed at least once prior to proceeding to Step IV of the preparation.

Prior to proceeding to Step IV, it is preferred that all bacteriological and serological testing be assessed to ensure that all tests are negative.

Step IV. The final step in this embodiment of the method of collagen biofabric production comprises drying the decellularized amniotic membrane of the invention to produce the collagen biofabric. Any method of drying the amniotic membrane so as to produce a flat, dry sheet of collagen may be used. Preferably, however, the amniotic membrane is dried under vacuum.

In a specific embodiment, an exemplary method for drying the decellularized amniotic membrane of the invention comprises the following steps:

Assembly of the decellularized amniotic membrane for drying. The decellularized amniotic membrane is removed from the sterile solution, and the excess fluid is gently squeezed out. The decellularized amniotic membrane is then gently stretched until it is flat with the fetal side faced in a downward position, e.g., on a tray. The decellularized amniotic membrane is then flipped over so that fetal side is facing upwards, and placed on a drying frame, preferably a plastic mesh drying frame (e.g., Quick Count® Plastic Canvas, Uniek, Inc., Waunakee, Wis.). In other embodiments, the drying frame may be any autoclavable material, including but not limited to a stainless steel mesh. In a most preferred embodiment, about 0.5 centimeter of the amniotic membrane overlaps the edges of the drying frame. In certain embodiments, the overlapping amniotic membrane extending beyond the drying frame is wrapped over the top of the frame, e.g., using a clamp or a hemostat. Once the amniotic membrane is positioned on the drying frame, a sterile gauze is placed on the drying platform of a heat dryer (or gel-dryer) (e.g., Model 583, Bio-Rad Laboratories, 200 Alfred Nobel Drive, Hercules, Calif. 94547), so that an area slightly larger than the amniotic membrane resting on the plastic mesh drying frame is covered. Preferably, the total thickness of the gauze layer does not exceed the thickness of one folded 4×4 gauze. Any heat drying apparatus may be used that is suitable for drying sheet like material. The drying frame is placed on top of the gauze on the drying platform so that the edges of the plastic frame extend above beyond the gauze edges, preferably between 0.1-1.0 cm, more preferably 0.5-1.0 cm. In a most preferred embodiment, the drying frame having the amniotic membrane is placed on top of the sterile gauze with the fetal side of the amniotic membrane facing upward. In some embodiments, another plastic framing mesh is placed on top of the amniotic membrane. A view of the mesh frame and the membrane dried therein is shown in FIG. 4. In another embodiments, a sheet of thin plastic (e.g., SW 182, clear PVC, AEP Industries Inc., South Hackensack, N.J. 07606) or a biocompatible silicone is placed on top of the membrane covered mesh so that the sheet extends well beyond all of the edges. In this embodiment, the second mesh frame is not needed.

In an alternative embodiment, the amniotic membrane is placed one or more sterile sheets of Tyvek® material (e.g., a sheet of Tyvek® for medical packaging, Dupont Tyvek®, P.O. Box 80705, Wilmington, Del. 19880-0705), optionally, with one sheet of Tyvek® on top of the membrane (prior to placing the plastic film). This alternate process will produce a smoother version of the biofabric (i.e., without the pattern of differential fiber compression regions along and perpendicular to the axis of the material), which may be advantageous for certain applications, such as for example for use as a matrix for expansion of cells.

Drying the amniotic membrane. In a preferred embodiment, the invention encompasses heat drying the amniotic membrane of the invention under vacuum. While the drying under vacuum may be accomplished at any temperature from about 0° C. to about 60° C., the amniotic membrane is preferably dried at between about 35° C. and about 50° C., and most preferably at about 50° C. It should be noted that some degradation of the collagen is to be expected at temperatures above 50° C. The drying temperature is preferably set and verified using a calibrated digital thermometer using an extended probe. Preferably, the vacuum pressure is set to about −22 inches of Hg. The drying step is continued until the collagen matrix of the amniotic membrane is substantially dry, that is, contains less than 20% water by weight, and preferably, about 3-12% water by weight as determined for example by a moisture analyzer. To accomplish this, the amniotic membrane may be heat-vacuum dried, e.g., for approximately 60 minutes to achieve a dehydrated amniotic membrane. In some embodiments, the amniotic membrane is dried for about 30 minutes to 2 hours, preferably about 60 minutes. Although not intending to be bound by any mechanism of action, it is believed that the low heat setting coupled with vacuum pressure allows the amniotic membrane to achieve the dehydrated state without denaturing the collagen.

After completion of the drying process in accordance with the invention, the amniotic membrane is cooled down for approximately two minutes with the vacuum pump running.

Packaging and Storing of the Amniotic Membrane. Once the amniotic membrane is dried, the membrane is gently lifted off the drying frame. “Lifting off” the membrane may comprise the following steps: while the pump is still running, the plastic film is gently removed from the amniotic membrane starting at the corner, while holding the amniotic membrane down; the frame with the amniotic membrane is lifted off the drying platform and placed on a cutting board with the amniotic membrane side facing upward; an incision is made, cutting along the edge 1-2 mm away from the edge of the frame; the amniotic membrane is then peeled off the frame. Preferably, handling of the amniotic membrane at this stage is done with sterile gloves.

The amniotic membrane is placed in a sterile container, e.g., peel pouch, and is sealed. The biofabric produced in accordance with the methods of the invention may be stored at room temperature for an extended period of time as described supra.

In alternative embodiments, the invention provides a method of preparing a collagen biofabric comprising a chorionic membrane, or both a chorionic membrane and an amniotic membrane. It is expected that the methods described above would be applicable to the method of preparing a biofabric comprising a chorionic membrane, or both a chorionic membrane and an amniotic membrane. In one embodiment, the invention encompasses the use of a collagen biofabric prepared by providing a placenta comprising an amniotic membrane and a chorionic membrane; separating the amniotic membrane from the chorionic membrane; and decellularizing the chorionic membrane. In a specific embodiment, the method further entails washing and drying the decellularized chorionic membrane. In another embodiment, the invention encompasses the use of a collagen biofabric prepared by providing a placenta comprising an amniotic membrane and a chorionic membrane, and decellularizing the amniotic and chorionic membranes. In a specific embodiment, the method further entails washing and drying the decellularized amniotic and chorionic membranes.

5.2.2 Storage and Handling of Collagen Biofabric

Dehydrated collagen biofabric may be stored, e.g., as dehydrated sheets, at room temperature (e.g., 25° C.) prior to use. In certain embodiments, the collagen biofabric can be stored at a temperature of at least 10° C., at least 15° C., at least 20° C., at least 25° C., or at least 29° C. Preferably, collagen biofabric, in dehydrated form, is not refrigerated. In some embodiments, the collagen biofabric may be refrigerated at a temperature of about 2° C. to about 8° C. The biofabric produced according to the methods of the invention can be stored at any of the specified temperatures for 12 months or more with no alteration in biochemical or structural integrity (e.g., no degradation), without any alteration of the biochemical or biophysical properties of the collagen biofabric. The biofabric can be stored for several years with no alteration in biochemical or structural integrity (e.g., no degradation), without any alteration of the biochemical or biophysical properties of the collagen biofabric. The biofabric may be stored in any container suitable for long-term storage. Preferably, the collagen biofabric of the invention is stored in a sterile double peel-pouch package.

Once formed, ocular plugs formed from collagen biofabric may be stored in the same manner as the collagen biofabric. Ocular plugs are preferably stored dry at a temperature of at least 10° C., at least 15° C., at least 20° C., at least 25° C., or at least 29° C. Ocular plugs may also be stored in a sterile, physiologically-acceptable solution, e.g., 0.9% NaCl solution, prior to use.

5.2.3 Sterilization

Sterilization of the biofabric may be accomplished by any medically-appropriate means, preferably means that do not significantly alter the tertiary and quaternary structure of the amniotic membrane proteins. Sterilization may be accomplished, for example, using gas, e.g., ethylene dioxide. Sterilization may be accomplished using radiation, for example, gamma radiation, and is preferably done by electron beam irradiation using methods known to one skilled in the art, e.g., Gorham, D. Byrom (ed.), 1991, Biomaterials, Stockton Press, New York, 55-122. Any dose of radiation sufficient to kill at least 99.9% of bacteria or other potentially contaminating organisms is within the scope of the invention. In a preferred embodiment, a dose of at least 18-25 kGy is used to achieve the terminal sterilization of the biofabric.

5.3 Making Ocular Plugs

The present invention further provides a method of making an ocular plug. The ocular plug of the present invention may be made by any method used to create or produce molded devices.

Ocular plugs may be made, for example, by stamping the plugs from a sheet of material using a shaped stamp. Alternatively, the plugs may be cut from a sheet of material, or may be formed by removal of unwanted material from a block of plug material. In a preferred embodiment, the ocular plug of the invention is formed using a mold. Where the ocular plug is formed using a mold, the plug material is preferably first made into a liquid, slurry, paste, or similar material amenable to forming in a mold.

In an exemplary embodiment of a method of making the ocular plug, the ocular plug is made of a biodegradable material, preferably collagen biofabric formed from the amnion of a post-partum mammalian placenta. The material is first reduced to a collection of particles; that is, the biofabric is micronized. The material may be micronized to a particle size of anywhere from 1 micron to millimeter. Generally, the larger the particle size, the more porous the plug. Any method may be used to micronize the biofabric, for example, ultrasound, physical shearing, homogenization, etc. Such micronization may be done dry (that is, by micronizing the biofabric without any additional liquid), or may be done using a micronization liquid or carrier. If micronization with a liquid is performed, the liquid may be any physiologically acceptable liquid or solution that does not significantly degrade the biodegradable material, e.g., the tertiary structure of the proteins comprising collagen biofabric. Typically, the ratio of biodegradable material to liquid is 25 mg/ml to 300 mg/ml, but more or less of the biofabric may be used. Determination that a desired particle size has been achieved may be accomplished by any means known in the art, e.g., microscopic examination, comparison to bead size standards, etc.

Once the desired micronized material is obtained, whether in wet or dry form, the micronized material is injected or otherwise forced into the mold and allowed to set. In one embodiment, the wet micronized material is forced into the mold and is then frozen, e.g., at a temperature of from −5° C. to −160° C. (though higher or lower temperatures would also work) for a time-sufficient to allow ice crystals to form and grow, e.g., 2 hours to several days. The frozen plug is then freeze dried to substantial dryness, that is, to a water content of about 20% by weight or less. Preferably, any plugs formed using a biodegradable material, particularly collagen biofabric, and particularly collagen biofabric micronized in a micronizing solution, are freeze-dried to substantial dryness. Freeze-drying is particularly preferred as the process allows for the development of pores in the material constituting the plug. Plugs may also be heat-dried, but heat applied to dry the plugs is preferably not heat that would cause the breakdown of any component of the plug material, e.g., collagen in a collagen biofabric, out of which the plug is made. For example, in various embodiments, an ocular plug formed from collagen biofabric may be dried at about 70° C., about 65° C., about 60° C., about 55° C., about 50° C. or about 45° C., or less than about 70° C., less than about 65° C., less than about 60° C., less than about 55° C., less than about 50° C. or less than about 45° C.

Once the plugs are freeze-dried, or dried by other method, the plug material is preferably cross-linked to provide mechanical stability and integrity. This is particularly important for a natural plug material, such as collagen biofabric, the structural integrity of which is disrupted during micronization. Crosslinking may be accomplished by any method known in the art; particularly preferred are radiation, chemical, or heat crosslinking.

Radiation crosslinking is preferred. Radiation used may be any known in the art to be useful for such a purpose, for example, electron-beam or e-beam radiation, gamma radiation or ultraviolet radiation. See, e.g., Odland, U.S. Pat. No. 5,989,498 “E-beam Sterilization of Biological Materials,” The intensity of radiation used may be that ordinarily used for the sterilization of medical instruments.

The material of the ocular plug may also be chemically crosslinked using any chemical crosslinking methodology known in the art, for example, thiol-thiol crosslinking, amide-amide crosslinking, amine-thiol crosslinking, amine-carboxylic acid and thiol-carboxylic acid crosslinking, etc., as appropriate for the material from which the plug is made.

The freeze-dried plug material may also be heat crosslinked, typically using a thermal dehydration process. Most preferably the heat used for such crosslinking does not significantly degrade or structurally weaken the material in any way. Plugs may be heat crosslinked, for example, by placing the plugs in a vacuum oven at 105° C. for 1-5 hours, or until the desired structural integrity or degree of crosslinking is achieved.

The ocular plug of the present invention may be formed into any shape that can substantially occlude or fill a discontinuity in the sclera, e.g., prevent leakage or infection through the discontinuity. Preferably the plug is formed to have a cylindrical shaft, with or without portions of the shaft that act as anchors, but may be made in the cross-sectional shape of a square, polygon, oval, triangle, or other geometric shape; additionally, the cross-section of the shaft may be irregular to conform to the particular dimensions of a particular scleral hole or discontinuity. For example, in one embodiment of the method of making the ocular plug, photomicrographs of the scleral discontinuity are taken, as an adjunct to ocular surgery, or as an adjunct to scleral repair, and the appropriate cross-sectional shape or conformation of a plug is determined. An appropriate ocular plug would then be custom-made.

The ocular plugs of the invention may be pre-made to standard sizes, or may be custom-made to fill particular scleral holes or discontinuities, as discussed above in Section 5.1.

The ocular plugs of the invention may also be coated or impregnated during manufacture with one or more of the bioactive or medicinal compounds disclosed above in Section 5.2.1.1.

5.4 Uses of Ocular Plugs

5.4.1 Scleral Discontinuity Occlusion and Repair

The present invention also provides methods of using the ocular plugs, described above, e.g., in the occlusion and repair of discontinuities in the sclera of an individual. Such discontinuities may be substantially circular holes, such as injection holes, surgical holes of any shape or size, or may be discontinuities caused by accident, trauma or injury. The individual may be any mammal, for example, domestic animals such as dogs or cats; livestock such as horses, cattle, swine, sheep, goats, buffalo, llama, etc., but is preferably human. Preferably the material used to make the ocular plug, if from a natural source, is made from a source that is the same species as the recipient individual.

The present invention provides a method of occluding a discontinuity, e.g., a hole, in the sclera of an individual comprising inserting an ocular plug into the discontinuity so as to substantially seal the discontinuity, wherein the ocular plug is made from a biodegradable composition. In a specific embodiment, said plug comprises a shaft and optionally a cap, the shaft having a length and two ends, said shaft extending from the cap. In another embodiment, the ocular plug comprises a shaft the dimensions of which exceed the size of the discontinuity. In a preferred embodiment, the ocular plug, when inserted into the discontinuity, inhibits leakage of ocular fluid from the eye, and inhibits bacteria from entering the eye.

In another specific embodiment, the discontinuity is a needle hole or a hole formed in the sclera to allow passage of one or more surgical instruments into the interior of the eye. In a specific embodiment, the needle or hole is a standard size, and the plug is designed to occlude a hole of that size. In another specific embodiment, the discontinuity is irregular in shape, and the ocular plug is shaped to occlude the discontinuity. In a specific embodiment, the discontinuity is intentionally-made, e.g., in preparation for surgery or by injection into the eye. In a more specific embodiment, the discontinuity is a hole in the sclera left after passage of a needle. In another more specific embodiment, said discontinuity is a hole created in the sclera to allow passage of one or more surgical instruments. In another more specific embodiment, said discontinuity is a slit.

In another embodiment, the discontinuity is created through trauma, injury or accident, and may be a puncture, rip, tear, or similar discontinuity. In a specific embodiment, said discontinuity is occluded by two or more ocular plugs.

A particularly useful aspect of the ocular plugs of the present invention is that they are constructed of a biodegradable material, e.g., collagen biofabric, such that the plugs need not be removed after insertion.

Preferably, the plugs also facilitate the ingrowth of scleral cells to facilitate healing of the hole or other discontinuity. The invention therefore comprises a method of repairing a discontinuity in the sclera of an individual, comprising occluding the discontinuity with an ocular plug made from a biodegradable material, wherein said ocular plug encourages or facilitates the regrowth of the sclera into the discontinuity.

The ocular plugs of the invention may be inserted into the sclera of the eye by any medically-acceptable means. Typically, the plug is inserted using a medical instrument under a surgical microscope. In a preferred embodiment, wherein the ocular plug has an elongated opening extending at least partway through the cap and, optionally, into the shaft, the medical instrument is a wire of fixed gauge, e.g., 19 gauge. In this embodiment, the wire is inserted into the longitudinal opening, and the ocular plug is inserted, e.g., into the sclera using the wire as a guide. In another embodiment, the surgical instrument is a forceps, for example, a blunt-tip forceps such as the one described in U.S. Pat. No. 6,846,318.

5.4.2 Drug Delivery

The ocular plug of the present invention may also, in addition to or alternatively to use in scleral repair, be used for the purpose of drug delivery, either to the site of a discontinuity already present in the sclera or to another part of the eye, e.g., the retina, macula, optic nerve, etc. Thus, the present invention provides a method of delivering a drug to the eye comprising implanting within the sclera of said eye an ocular plug comprising a bioactive compound. In one embodiment, said ocular plug is made of one or more biodegradable components. In another embodiment, said ocular plug is coated or impregnated with at least one bioactive compound. In a specific embodiment, said bioactive compound is delivered into the vitreous humor or interior of the eye. In another specific embodiment, said bioactive compound is delivered primarily to the sclera. In another specific embodiment, said bioactive compound is delivered to the back of the eye, e.g., to the retina, macula or optic nerve. In another specific embodiment, said bioactive compound is an antibiotic, antiviral agent, antimicrobial agent, anti-inflammatory agent, antiproliferative agent, cytokine, enzyme or protein inhibitor, or antihistamine. In another specific embodiment, said ocular plug is inserted into the sclera primarily to deliver a drug to the sclera or to the eye, rather than to repair an ocular hole caused by a separate medical procedure, trauma, accident or blow. In another specific embodiment, said ocular plug is coated or impregnated with at least two bioactive compounds. In another specific embodiment, said ocular plug comprising said bioactive molecule is implanted so as to float within the vitreous humor.

The amount of the bioactive compound coating or impregnating the ocular plug may vary, and will preferably depend upon the particular bioactive compound to be delivered, and the effect desired. For example, where the bioactive compound is an anti-inflammatory agent, the amount of the anti-inflammatory agent on or contained by the ocular plug is an amount sufficient to measurably reduce one or more symptoms or indicia of inflammation in the sclera, if reduction of inflammation in the sclera adjacent to the ocular plug is the desired effect, or in the eye generally, if reduction of inflammation in eye structures or tissues in addition to the sclera is desired.

In various embodiments, the ocular plug of the invention may be coated with, or impregnated with, at least 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound. In another embodiment, the ocular plug of the invention may be coated with, or impregnated with, no more than 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound.

In various other embodiments, the ocular plugs of the invention may be coated or impregnated with antibiotics, antiemetic agents, antidepressants, and antifungal agents, anti-inflammatory agents, antiviral agents, immunomodulatory agents, interferons, alkylating agents, hormones or cytokines, or any of the compounds listed above in Section 5.2.4.

5.4.3 Lachrymal Plugs

The ocular plug of the present invention may also be used as a lachrymal or punctum plug for occluding the lachrymal canal. The ocular plug, when used to occlude the lachrymal canal, preferably the flow of lachrymal fluid, is preferably designed to fit within a wide range of differently sized puncta. In various embodiments, for example, the plug is preferably 1.5 mm to 2.5 mm long from head to tip; the shaft is preferably 1.4 mm to 2.4 mm long, and 0.4 mm to 0.6 mm in diameter; and the head is preferably 1.5 mm to 2.5 mm in diameter (that is, a diameter which is larger than a diameter of the punctal opening of a recipient of the plug).

5.5 Stem Cells

The ocular plugs described herein may also comprise stem or progenitor cells. In one embodiment, the ocular plug is a delivery device for one or more stem or progenitor cells. The ocular plug may comprise any kind of stem or progenitor cell. Preferably, the ocular plug comprises limbal stem cells, or placenta-derived stem cells such as those described in U.S. Pat. No. 7,045,148, and U.S. Application Publication Nos. 2003/0032179 and 2003/0180269. However, the collagen biofabric may comprise stem or progenitor cells, preferably mammalian stem or progenitor cells, from any tissue source. The collagen biofabric may comprise embryonic stem cells or embryonic germ cells.

The combination of ocular plug and stem or progenitor cells may be accomplished prior to or during application of the ocular plug to the eye. For example, an ocular plug can be prepared immediately prior to application to the eye by disposing on the surface of the ocular plug a solution of stem or progenitor cells, and allowing the stem or progenitor cells sufficient time to adhere to the ocular plug. The stem or progenitor cells, alternately, may be disposed onto the surface of the ocular plug 30 minutes, 1, 2, 3, 4, 5, 6, 7, 8, 9, 10, 12, 14, 16, 18, 20, 22, 24 or more hours prior to application of the ocular plug to the eye. The number of stem or progenitor cells disposed onto the surface of the ocular plug may vary, but may be at least 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; or may be no more than 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹² stem or progenitor cells. In a more specific embodiment, the stem cells are applied in a physiologically-acceptable liquid, such as a saline solution, or embedded in a physiologically-acceptable gel, such as a hydrogel, in which the stem or progenitor cells may be maintained and migrate through. The stem cells, prior to or after contacting with the ocular plug, may be contacted with one or more differentiation-modulating agents, for example, the differentiation-modulating agents described in U.S. Application Publication Nos. 2003/0235909, 2004/0028660, or International Application Publication No. WO 03/087333. Methods of differentiating stem cells to, for example, epidermal, mesodermal, and other cell types are known in the art, and are described, e.g., in U.S. Application Publication No. 2004/0028660.

5.6 Kits

The invention further comprises kits providing one or more of the ocular plugs of the invention in a suitable container. Kits of the invention comprise one or more ocular plugs, and may comprise other components, such as an instrument for inserting the ocular plug into the sclera, one or more bioactive compounds in one or more separate containers, one or more syringes, sterile gauze, gloves or other disposables, and the like.

A kit of the invention may comprise a single ocular plug, sterilely wrapped and ready for immediate use. In one embodiment, said ocular plugs are provided in substantially dry form. In another embodiment, said ocular plug is provided in a sterile liquid, e.g., a sterile saline solution. In other embodiments, a kit of the invention may comprise two or more ocular plugs of the same size. In another embodiment, the invention provides a kit comprising a syringe suitable for intraocular drug delivery and one or more ocular plugs sized to occlude the scleral hole left by said syringe. In a specific embodiment, said ocular plug comprises one or more bioactive compounds. In a more specific embodiment, said bioactive compound is an antihistamine, antimicrobial agent, antibiotic, antiviral agent, pain medication, anti-inflammatory agent, antiproliferative agent, cytokine, growth factor, or enzyme or kinase inhibitor. In another specific embodiment, said ocular plug and said bioactive compound are provided separately within said kit, in a form suitable for combining immediately prior to use. In this embodiment, the kit provides a container for allowing the user to coat or impregnate the ocular plug with said bioactive compound. In another embodiment, the kit provides two or more ocular plugs and two or more bioactive compounds in separate containers within said kit. In another embodiment, said kit provides ocular plugs of the same size in lots of 5, 10, 15, 20, 25, 30, 40, 50, 75, 100 or more. In another embodiment, said kit comprises two ocular plugs of different sizes. In a specific embodiment, said kit comprises ocular plugs suitable for occluding scleral holes formed during ocular surgery to allow the passage of surgical instruments into the eye, and for occluding holes caused by injections adjunct to said surgery. In another embodiment, said kit comprises an ocular plug of the invention and a surgical instrument suitable for placing the ocular plug into a hole in the sclera of the eye. In a specific embodiment, said surgical instrument is a forceps. In a more specific embodiment, said forceps is the forceps described in U.S. Pat. No. 6,846,318. In another more specific embodiment, the surgical instrument is a wire of fixed gauge, e.g., 19 gauge. In a more specific embodiment, the wire is made of any sufficiently stiff material, e.g., steel, aluminum, plastic, etc. In another more specific embodiment, the kit provides the wire attached to the ocular plug. In another embodiment, said kit comprises one or more containers of eyedrops. In a specific embodiment, said eyedrops comprise an antibiotic, an anti-inflammatory agent or a pain medication.

### 6. EXAMPLES

### 6.1 Example 1

### Method of Making Collagen Biofabric Materials

The following materials were used in preparation of the collagen biofabric.

Materials/Equipment

Copy of Delivery Record
Copy of Material/Family Health History/Informed Consent
Source Bar Code Label (Donor ID number)
Collection # (A sequential number is assigned to incoming material)
Tissue Processing Record (Document ID #ANT-19F); a detailed record of processing of each lot number is maintained
Human Placenta (less than 48 hours old at the start of processing)
Sterile Surgical Clamps/Hemostats
Sterile Scissors
Sterile Scalpels
Steri-Wrap sheets
Sterile Cell Scraper (Nalgene NUNC Int. R0896)
Sterile Gauze (non-sterile PSS 4416, sterilized)
Sterile Rinsing Stainless Steel Trays
Disinfected Processing Stainless Steel Trays
Disinfected Plastic Bin
Sterile 0.9% NaCl Solution (Baxter 2F7124)
Sterile Water (Milli Q plus 09195 or Baxter 2F7113)
Sterile Specimen Containers (VWR 15704-014)
Personal Protective Equipment (including sterile and non-sterile gloves)
Certified Clean Room
Previously Prepared Decellularizing Solution (D-cell); 0.01-1% deoxycholic acid sodium monohydrate
Disinfected Bin
Rocking Platform (VWR Model 100)
Timer (VWR 21376890)
Disinfected Plastic Frame Mesh
PVC Wrap Film
Vacuum Pump (Schuco-Vac 5711-130)
Gel Dryer (i.e., heat dryer; BioRad Model 583)
Disinfected Stainless Steel Cutting Board
Pouches for Packaging
Sterile Stainless Steel Ruler (General Tools MFG. Co 1201)
Traceable Digital Thermometer (Model 61161-364, Control Company)
Accu-Seal Automatic Sealer (Accu-Seal, Model 630-1B6)

The expectant mother was screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens were used to produce the collagen biofabric.

Following normal birth, the placenta, umbilical cord and umbilical cord blood were spontaneously expelled from the contracting uterus. The placenta, umbilical cord, and umbilical cord blood were collected following birth. The materials were transported to the laboratory where they were processed under aseptic conditions in a Clean room having a HEPA filtration system, which was turned on at least one hour prior to processing. Gloves (sterile or non-sterile, as appropriate) were worn at all times while handling the product. All unused (waste) segments of the amnion/chorion and contaminated liquids generated during tissue processing were disposed of as soon as feasible.

Step I.

A sterile field was set up with sterile Steri-Wrap sheets and the following instruments and accessories for processing were placed on it.
sterile tray pack
sterile Cell Scraper
sterile scalpel
disinfected processing tray

Sterile pack ID # was recorded in the Processing Record.

The placenta was removed from the transport container and placed onto the disinfected stainless steel tray. Using surgical clamps and scissors, the umbilical cord was cut off approximately 2 inches from the placental disc. The umbilical cord was placed into a separate sterile container for further processing. The container was labeled with Tissue ID Bar Code; and the material and storage solution(s) present (e.g., type of media) were identified. In some cases, the umbilical cord was discarded if not requested for other projects.

Starting from the edge of the placental membrane, the amnion was separated from the chorion using blunt dissection with fingers. This was done prior to cutting the membrane.

After the amnion was separated from the entire surface of the chorion and placental disc, the amniotic membrane was cut around the umbilical cord stump with scissors and detached from the placental disc. In some instances, if the separation of the amnion and chorion was not possible without tearing the tissue, the amnion and chorion were cut from the placental disc as one piece and then peeled apart.

The chorion was placed into a separate specimen container to be utilized for other projects. The container was labeled with the Tissue ID Bar Code, the material and storage solution(s) present (e.g., type of media) were identified, initialed and dated.

If any piece of amnion was still attached to the placental disc it was peeled from the disc and cutting off around the umbilical cord with scissors. The placenta was placed back into the transport container to be utilized for other projects.

The appropriate data was recorded in the Tissue Processing Record.

The amniotic membrane was kept in the tray with sterile 0.9% NaCl solution. Preferably, the amniotic membrane is stored by refrigeration for a maximum of 72 hours from the time of delivery prior to the next step in the process.

Step II.

The amniotic membrane was removed from the specimen container one piece at a time and placed onto the disinfected stainless steel tray. Other pieces were placed into a separate sterile stainless steel tray filled with sterile water until they were ready to be cleaned. Extra pieces of amnion from the processing tray were removed and placed in a separate rinsing stainless steel tray filled with sterile water.

The amniotic membrane was rinsed with sterile water if grossly contaminated with blood maternal or fetal fluids/materials changing sterile water as needed.

The amniotic membrane was placed on the processing tray with the maternal side facing upward. Using a sterile Cell Scraper, as much as possible of visible contamination and cellular material from the maternal side of the amnion was carefully removed. (Note: minimal pressure should be applied for this step to prevent tearing the membrane). Sterile water was used to aid in the removal of cells and cellular debris. The amniotic membrane was further rinsed with sterile water in the separate sterile stainless steel rinsing tray.

The amniotic membrane was turned over so that the fetal side was facing upward and placed back on the processing tray and rinsed with sterile water. Visible cellular material and debris using the Cell Scraper was gently removed (Note: minimal pressure should be applied for this step to prevent tearing the membrane). Sterile water was used to aid in the removal of cells and cellular debris.

The amniotic membrane was rinsed with sterile water in between cleaning rounds in separate sterile rinsing trays. The tissue was cleaned as many times (cleaning rounds) as necessary to remove most if not all of visible cellular material and debris from both sides of the membrane. The sterile water was changed in the rinsing trays in between rinses.

The processing tray was rinsed with sterile water after each cleaning round.

All other pieces of amnion were processed in the same manner and placed into the same container. Tissue Id Bar Code was affixed, the material and storage solution(s) present (e.g., type of media) were identified, initials date were added.

The appropriate information and the date were recorded in the Tissue Processing Record.

Step III.

The amniotic membrane was removed from the rinsing tray, (or from storage container) excess fluid was gently squeezed out with fingers and the membrane was placed into the sterile specimen container. The container was filled up to the 150 ml mark with D-cell solution ensuring that all of the amniotic membrane was covered and the container was closed.

The container was placed in the bin on the rocking platform. The rocking platform was turned on and the membrane was agitated in D-cell solution for a minimum of 15 minutes and a maximum of 120 minutes at Setting #6.

A new sterile field was set up with new sterile instruments and disinfected tray in a same manner as in the Step I. Sterile pack ID # was recorded in the Processing Record.

After agitation was completed, the rocking platform was turned off and the membrane was removed from the container. The membrane was placed into a new sterile stainless steel processing tray. Sterile 0.9% NaCl solution was added to cover the bottom of the tray.

Using a new sterile Cell Scraper, residual D-cell and cellular material (if any) was removed from both sides of the tissue. This step was repeated as many times as needed to remove as much as possible of visible residual cellular material from the entire surface on both sides. The membrane was rinsed with sterile 0.9% NaCl solution in a separate rinsing tray in between cleaning rounds. The sterile 0.9% NaCl solution was changed in the rinsing trays in between rinses.

After the last cleaning round was completed, the membrane was rinsed with sterile 0.9% NaCl solution and placed into the new sterile specimen container filled with sterile 0.9% NaCl solution.

All remaining pieces of amniotic membrane were processed in exactly the same manner.

When all amniotic membrane pieces were processed and in the container with the sterile 0.9% NaCl solution, the container was placed in the bin on the rocking platform to agitate for a minimum of 5 minutes at setting #6. After agitation was completed, the membrane was removed from the specimen container, the sterile 0.9% NaCl solution was changed in the container and the membrane was placed back into the specimen container.

The specimen container was labeled with Tissue ID Bar Code and Quarantine label. The material and storage solution(s) present (e.g., type of media) were identified, initialed and dated. The specimen container was placed into a clean zip-lock bag and placed in the refrigerator (2-8° C.).

All appropriate data was recorded in the Tissue Processing Record.

When serology results became available, the appropriate label (Serology Negative or For Research Use Only) was placed on the top of the Quarantine label and those containers were segregated from Quarantined ones.

Step IV.

Before proceeding with Step IV, the Tissue Status Review was checked to make sure all applicable test results were negative.

A sterile field was set up with sterile Steri-Wrap sheet and all sterile and disinfected instruments and accessories were set up in the same manner as in Steps II and III.

The membrane was removed from the refrigerator and placed into a new sterile stainless steel processing tray. Sterile 0.9% NaCl solution was added to cover the bottom of the tray.

All visible cellular material and debris (if any) was gently removed using a new sterile Cell Scraper (Note: minimal pressure should be applied for this step to prevent tearing the membrane). Sterile 0.9% NaCl solution was used to aid in removal of the cells and debris.

The membrane was rinsed in the separate sterile stainless steel rinsing tray filled with the sterile 0.9% NaCl Solution. 0.9% NaCl Solution was changed in between cleaning rounds. The membrane was placed into a new sterile specimen container, the container was filled with fresh sterile 0.9% NaCl solution and placed on the rocking platform for agitation for a minimum of 5 minutes at Setting #6.

The previous step was repeated 3 times and the sterile 0.9% NaCl solution was changed in between each agitation. Appropriate data was recorded in the Tissue Processing Record.

The membrane was removed from the specimen container one piece at a time, excess fluid was gently squeezed out with fingers and the membrane was placed onto a sterile processing tray. The membrane was gently stretched until flat; ensuring it was fetal side down.

The frame was prepared by cutting the disinfected plastic sheet with sterile scissors. The size of the frame should be approximately 0.5 cm smaller in each direction than the membrane segment. The frame was rinsed in the rinsing tray filled with sterile 0.9% NaCl solution.

The frame was placed on the slightly stretched membrane surface and pressed on it gently. It is imperative that the smooth side of the plastic frame faces the tissue.

Using a scalpel, the membrane was cut around the frame leaving approximately 0.5 cm extending beyond frame edges. The excess membrane was placed back into the specimen container

The membrane edges that are extended beyond the frame were wrapped over the edges of the frame using clamps or tweezers and put aside on the same tray.

The next piece of membrane was processed in the same manner. It is important the total area to be dried does not exceed 300 cm² per heat dryer. While ‘framing out’ the piece of membrane, the non-framed pieces should remain in the container in sterile 0.9% NaCl solution.

The drying temperatures of dryers were set and verified using a calibrated digital thermometer with extended probe. The drying temperature was set at 50° C. The data was recorded in the Tissue Processing Record.

The vacuum pump was turned on.

A sterile gauze was placed on the drying platform of the heat dryer, covering an area slightly larger than the area of the framed membrane. It is important to make sure that the total thickness of the gauze layer does not exceed thickness of one folded 4×4 gauze.

One sheet of plastic framing mesh was placed on top of the gauze. The plastic mesh edges should extend approximately 0.5-1.0 cm beyond gauze edges.

The framed membrane was gently lifted and placed on the heat dryer platform on top of the plastic mesh with the membrane side facing upward. This was repeated until the maximum amount of membrane (without exceeding 300 cm²) was on the heat dryer platform. (NOTE: fetal side of the amnion is facing up).

A piece of PVC wrap film was cut large enough to cover the entire drying platform of the heat dryer plus an extra foot.

With the vacuum pump running, the entire drying platform of the heat dryer was gently covered with the plastic film leaving ½ foot extending beyond drying platform edges on both sides. Care was taken that the film pull tightly against the membrane and frame sheet (i.e., it is “sucked in” by the vacuum) and that there were no air leaks and no wrinkles over the tissue area). The lid was subsequently closed.

The vacuum pump was set to approximately −22 inches Hg of vacuum. The pump gage was recorded after 2-3 min of drying cycle. The membrane was heat vacuum dried for approximately 60 minutes. Approximately 15-30 minutes into the drying process, the sterile gauze layer was replaced in the heat dryer with a new one. The total thickness of the gauze layer must not exceed thickness of one folded 4×4 gauze.

After the change, care was taken so that the plastic film pulled tightly against the membrane and the frame sheet and there were no air leaks and no wrinkles over the membrane area.

The integrity of the vacuum seal was periodically checked by checking the pump pressure monometer. After completion of the drying process, the heat dryer was opened and the membrane was cooled down for approximately two minutes with the pump running.

A new sterile field was set up with sterile Steri-wrap and disinfected stainless steel cutting board underneath it. As this point sterile gloves were used. With the pump still running, the plastic film was gently removed from the membrane sheet starting at the corner and holding the membrane sheet down with a gloved hand. The frame was gently lifted with the membrane off the drying platform and placed on the sterile field on the top of the disinfected stainless steel cutting board with the membrane side facing upward. Using a scalpel, the membrane sheet was cut through making an incision along the edge 1-2 mm away from the edge of the frame. The membrane was held in place with a gloved (sterile glove) hand. Gently the membrane sheet was lifted off of the frame by peeling it off slowly and then placed on the sterile field on the cutting board.

Using scalpel or sharp scissors, the membrane sheet was cut into segments of specified size. All pieces were cut and secured on the sterile field before packaging. A single piece of membrane was placed inside the inner peel-pouch package with one hand (sterile) while holding the pouch with another hand (non-sterile). Care was taken not to touch pouches with ‘sterile’ hand. After all pieces were inside the inner pouches they were sealed. A label was affixed with the appropriate information (e.g., Part #, Lot #, etc.) in the designated area on the outside of the pouch. All pieces of membrane were processed in the same manner. The labeled and sealed peel-pouch packages were placed in the waterproof zip-lock bag for storage until they were ready to be shipped to the sterilization facility or distributor. All appropriate data were recorded on the Tissue Processing Record.

### 6.2 Example 2

### Alternative Method of Making Collagen Biofabric

A placenta is prepared substantially as described in Step I of Example 1 using the Materials in that Example. An expectant mother is screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens are used to produce the collagen biofabric.

A sterile field is set up with sterile Steri-Wrap sheets and the following instruments and accessories for processing were placed on it: sterile tray pack; rinsing tray, stainless steel cup, clamp/hemostats, tweezers, scissors, gauze.

The placenta is removed from the transport container and placed onto a disinfected stainless steel tray. Using surgical clamps and scissors, the umbilical cord is cut off approximately 2 inches from the placental disc.

Starting from the edge of the placental membrane, the amnion is separated from the chorion using blunt dissection with fingers. This is done prior to cutting the membrane. After the amnion is separated from the entire surface of the chorion and placental disc, the amniotic membrane is cut around the umbilical cord stump with scissors and detached from the placental disc. In some instances, if the separation of the amnion and chorion is not possible without tearing the tissue, the amnion and chorion is cut from the placental disc as one piece and then peeled apart.

The appropriate data is recorded in the Tissue Processing Record.

The amniotic membrane is rinsed with sterile 0.9% NaCl solution to remove blood and fetal fluid or materials. The saline solution is replaced as necessary during this rinse.

The amnion is then placed in a 0.9% saline, 1.0% deoxycholic acid solution in a specimen container and refrigerated at 2-8° C. for up to 15 days, with changes of the solution every 3-5 days. During or at the end of incubation, the serological tests noted above are evaluated. If the tests indicate contamination with one or more pathogens, the amnion is rejected and processed no further. Tissue indicated as derived from a CMV-positive donor, however, is still suitable for production of biofabric.

Once the incubation is complete, the amnion is removed from the specimen container, placed in a sterile tray and rinsed three times with 0.9% NaCl solution to reduce the deoxycholic acid from the tissue. With the amnion placed maternal side up, the amnion is gently scraped with a cell scraper to remove as much cellular material as possible. Additional saline is added as needed to aid in the removal of cells and cellular debris. This step is repeated for the fetal side of the amnion. Scraping is followed by rinsing, and is repeated, both sides, as many times as necessary to remove cells and cellular material. The scraped amnion is rinsed by placing the amnion in 0.9% saline solution a separate container on a rocking platform for 5-120 minutes at setting #6. The saline solution is replaced, and the rocking rinse is repeated.

After rinsing is complete, the amnion is optionally stored in a zip-lock bag in a refrigerator.

The scraped amnion is then placed fetal side down onto a sterile processing tray. The amnion is gently massaged by hand to remove excess liquid, and to flatten the membrane. A sterile plastic sheet is cut so that its dimensions are approximately 0.5 cm smaller in each direction than the flat amnion. This plastic sheet is briefly rinsed in 0.9% NaCl solution. The plastic sheet is placed, smooth side down, on the flattened amnion, leaving a margin of uncovered amnion. A scalpel is used to trim the amnion, leaving approximately 0.5 cm extending beyond the sheet edges. These extending amnion edges are wrapped back over the plastic sheet. The total tissue area to be dried does not exceed 300 cm² for a standard vacuum heat dryer.

A sheet of sterile gauze is placed in a vacuum heat dryer. A thin plastic mesh is placed on the gauze so that approximately 0.5-10.0 cm extends beyond the edges of the gauze. The amnion and plastic sheet are then placed into the vacuum heat dryer on top of the mesh, tissue side up, and the amnion is covered with a sheet of PVC wrap film. The dryer is set at 50° C., and the temperature is checked periodically to ensure maintenance of 50° C.±1° C. The vacuum pump is then turned on and set to approximately −22 inches Hg vacuum. Drying is allowed to proceed for 60 minutes.

The dried amnion is then stored in a sealed plastic container for further use.

Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. An ocular plug comprising a cap and a shaft, the shaft having a length and two ends, said shaft extending from the cap, wherein said plug is made of a biodegradable composition.

2. The ocular plug of claim 2, wherein said biodegradable composition comprises dried amniotic membrane.

3. The ocular plug of claim 1, wherein said shaft comprises a narrow portion and a wide portion, wherein said wide portion has a greater cross-sectional area than said narrow portion.

4. The ocular plug of claim 2, wherein said narrow portion is proximal to said cap, and said wide portion is distal to said cap.

5. The ocular plug of claim 2, wherein said wide portion is disposed along the length of said shaft between said ends.

6. The ocular plug of claim 2, wherein said shaft has substantially equal cross-sectional area along its length.

7. The ocular plug of claim 1, wherein said cap is substantially flat.

8. The ocular plug of claim 1, wherein said plug is adapted for insertion into a hole in the sclera made as part of vitreo-retinal surgery or made by a needle.

9. The ocular plug of claim 1, wherein the cross-sectional area of said cap is greater than the cross-sectional area of said narrow portion of said shaft proximal to said cap.

10. The ocular plug of claim 1, wherein said plug comprises a compound that inhibits the growth of, or kills, one or more microorganisms.

11. The ocular plug of claim 1, wherein said plug is coated with a tissue adhesive.

12. A method of making an ocular plug, comprising:
(a) micronizing a dried amniotic membrane to produce micronized amniotic membrane;
(b) forming said micronized amniotic membrane in a mold to produce an amniotic membrane plug;
(c) freeze-drying said amniotic membrane plug to substantial dryness; and
(d) crosslinking said amniotic membrane plug to form an ocular plug.

13. The method of claim 12, wherein said micronizing is performed using a blender.

14. The method of claim 12, wherein the median size of particles in said micronized amniotic membrane is 1 micron to 1 millimeter.

15. The method of claim 12, wherein said freeze drying reduces the water content of said amniotic membrane plug to 20% or less by weight.

16. A method of occluding a discontinuity in a sclera of an eye comprising placing the ocular plug of claim 1 into the discontinuity such that the ocular plug occludes the discontinuity.

17. The method of claim 16 wherein the ocular plug inhibits leakage of fluid from the interior of the eye when placed in the discontinuity.

18. The method of claim 16 wherein said ocular plug comprises a bioactive compound.

19. The method of claim 16, wherein said discontinuity is a needle hole or a hole formed as part of ocular surgery.

20. The method of claim 16, wherein said discontinuity results from accident or trauma.
